(19) United States
(12) Patent Application Publication
(10) Pub. No.: US 2002/0061319 A1
(43) Pub. Date: May 23, 2002

(54) Film-forming cosmetic composition

(76) Inventors: Pascale Bernard, Sucy en Brie (FR); Xavier Blin, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/965,792

(22) Filed: Oct. 1, 2001

(30) Foreign Application Priority Data
Sep. 29, 2000 (FR) 00 12463

(51) Int. Cl. A61K 7/00

(52) U.S. Cl. 424/401

(57) ABSTRACT

A film-forming cosmetic composition comprising particles of at least one polymer in an aqueous dispersion, wherein the at least one polymer has a glass transition temperature (T_{g}) ranging from 35° C. to 80° C. and a minimum film-forming temperature (MFT) such that T_{g} −MFT≧8° C., and at least two organic solvents, where a first organic solvent has a molecular weight less than or equal to 200 and a boiling point ranging from 100° C. and 300° C., and a second organic solvent has a molecular weight greater than 200 and a boiling point greater than or equal to 120° C. The invention also relates to a cosmetic care or make-up process for keratinic materials.

The present invention relates to a film-forming cosmetic composition comprising an aqueous dispersion of at least one polymer and at least two organic solvents usable, for example, as a composition for making-up or care of keratinic materials such as skin, lips, nails, eyelashes, eyebrows and hair. In one embodiment of the invention the keratinic materials are of human origin. The invention relates also to the process of using the film-forming composition for the making-up or the cosmetic care of keratinic materials. In one embodiment of the invention the composition is a nail varnish.

Non-limiting examples of the utility of the nail varnish composition may include a base for varnishes, a product for making-up the nails, a finishing composition, also called “top-coat” in Anglo-American terminology, to be applied onto the nail make-up product, and a product for cosmetic care of the nails. These compositions can be applied onto the nails of human beings as well as false nails.

Film-forming cosmetic compositions, such as for example, nail varnishes, may, in certain embodiments, contain a polymer as film-forming agent and possibly coloring materials. These compositions may display at least one cosmetic property chosen from, for example, rapid film formation to give a film which is not sticky to the touch, is capable of resisting mechanical challenges such as impacts and rubbing, is capable of resisting solvents such as water, has a glossy appearance to impart a pleasant visual aesthetic effect; has adequate adherence to the keratinic materials, has sufficient durability with no alteration of the initial properties over the course of time, such as for example, after 2 days, and is capable of being easily removed with available standard removers, such as for example, acetone based and ethyl acetate based removers.

Aqueous cosmetic compositions comprising a polymer in an aqueous dispersion are known. Very often, the polymer has a glass transition temperature and film-forming temperature which are close to one another. However, with such a polymer, it is generally not possible to obtain a film which dries rapidly, is not sticky, displays good hardness, and is easy to remove with standard removers.

One embodiment of the present invention, therefore, is a film-forming cosmetic composition in aqueous medium, such as for example, a nail varnish, which forms a film with at least one desirable property, such as adhesion and glossiness, is non-sticky and can readily be cleaned off with standard removers based on at least one of, for example, acetone and ethyl acetate.

The inventors have found that such a composition may be obtained by combining at least one particular polymer in an aqueous dispersion with selected solvents.

Thus, one embodiment of the invention is a film-forming cosmetic composition comprising:

particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and

at least two organic solvents wherein:

a first organic solvent has a molecular weight less than or equal to 200 and a boiling point, measured at ambient pressure, ranging from 100° C. and 300° C., and

a second organic solvent has a molecular weight greater than 200 and a boiling point, measured at ambient pressure, greater than or equal to 120° C.

Another embodiment of the invention is a method for making-up or care of a keratinic material, for example nails, comprising:

applying to the keratinic materials a film-forming cosmetic composition comprising:

particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and

at least two organic solvents, wherein:

a first organic solvent has a molecular weight less than or equal to 200 and a boiling point, measured at ambient pressure, ranging from 100° C. and 300° C., and

a second organic solvent has a molecular weight greater than 200 and a boiling point, measured at ambient pressure, greater than or equal to 120° C.

The invention also relates to a method for forming a film comprising

applying to a keratinic material a film-forming composition comprising:

particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and

at least two organic solvents wherein:

a first organic solvent has a molecular weight less than or equal to 200 and a boiling point, measured at ambient pressure, ranging from 100° C. and 300° C., and

a second organic solvent has a molecular weight greater than 200 and a boiling point, measured at ambient pressure, greater than or equal to 120° C.,

wherein the film formed on the keratinic material has at least one of the following properties: is removable with at least one compound chosen, for example, from acetone and ethyl acetate; adheres to the nail; and is glossy.

In another embodiment, the invention relates to a film-forming composition comprising:

at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and

at least two organic solvents wherein:

a first organic solvent has a molecular weight less than or equal to 200 and a boiling point, measured at ambient pressure, ranging from 100° C. and 300° C., and

a second organic solvent has a molecular weight greater than 200 and a boiling point, measured at ambient pressure, greater than or equal to 120° C.

In yet another embodiment, the invention relates to a method for forming a film comprising applying to a keratinic material a film-forming composition comprising:

at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and

at least two organic solvents wherein:

a first organic solvent has a molecular weight less than or equal to 200 and a boiling point, measured at ambient pressure, ranging from 100° C. and 300° C., and

a second organic solvent has a molecular weight greater than 200 and a boiling point, measured at ambient pressure, greater than or equal to 120° C.,

wherein the film formed on the keratinic material has at least one of the following properties: is removable with at least one compound chosen, for example, from acetone and ethyl acetate; adheres to the nail; and is glossy.

For purposes of this invention, unless otherwise indicated, the term “molecular weight” is understood to mean the weight-average molecular weight.

In one embodiment of the invention, the at least one polymer in an aqueous dispersion present in the film-forming composition has at least one glass transition temperature (T_{g}) which ranges from 35° C. to 80° C. (even though the at least one polymer may have several T_{g}S) and a minimum film-forming temperature (MFT) such that T_{g}−MFT≧8° C.

The glass transition temperature is defined for the at least one polymer in an aqueous dispersion in the absence of auxiliary film-forming agents and is determined for a film dried at ambient temperature (23° C.) and at ambient relative humidity (50%).

In one embodiment of the invention, the glass transition temperature (T_{g}) of the at least one polymer ranges from 40° C. to 65° C.

Non-limiting examples of T_{g}−MFT are those chosen, for example, from T_{g}−MFT≧12° C., T_{g}−MFT≧16° C. and Tg−MFT≧18° C. In a further example, T_{g}−MFT is less than or equal to 25° C.

The at least one polymer in an aqueous dispersion according to the invention is characterized by its MFT in the absence of auxiliary film-forming agents and the glass transition temperature (T_{g}) of the film obtained after drying. The MFT is the limit temperature beyond which an aqueous dispersion of polymer forms a crack-free film on drying under predetermined conditions. The determination of the MFT is carried out according to the conditions described in DIN 53787, the disclosure of which relating to the determination of the MFT is specifically incorporated by reference, herein. At the start of the determination, the aqueous dispersion of the at least one polymer has a dry matter polymer content ranging from 30 to 55% by weight. For the purposes of the invention, the mean of 10 independently performed measurements is used as the MFT. The determination of the MFT is carried out in the absence of auxiliary film-forming agents, that is to say, the MFT is an intrinsic property of the polymer in dispersion. In addition, the formulated film-forming cosmetic composition in accordance with the invention may optionally comprise at least one auxiliary film-forming agent.

The determination of the glass transition temperature (T_{g}) is carried out in a normal commercial Differential Scanning Calorimeter (DSC calorimeter), with a heating rate of 20° C./minute for a dry film of 100 μm thickness dried at 23° C. and at a relative humidity level of 50%.

The size of the particles of the at least one polymer in an aqueous dispersion may range, for example, from 50 to 200 nm, such as for example, from 80 to 150 nm. Such particle size makes it possible to obtain a film which may exhibit high and durable gloss.

The at least one polymer in an aqueous dispersion is generally obtained by polymerization of at least one ethylenically unsaturated monomer in a two-phase system comprising a continuous aqueous phase. Typically, a water-soluble initiator system is used to initiate the polymerization. In general, the aqueous phase may contain at least one compound chosen from emulsifiers and protective colloid agents.

Non-limiting representatives of the at least one ethylenically unsaturated monomer may be chosen, for example, from (C₁-C₃₀) alkyl (meth)acrylates, vinylic esters, and aromatic vinylic monomers.

Non-limiting representatives of protective colloid agents may be chosen, for example, from hydrophilic polymers and hydrophilic copolymers, such as for example, polyvinyl alcohols, polyacrylic acids, polyacrylamides, polyvinylpyrrolidones, polyesters containing sulfonate groups, polyamides containing sulfonate groups, polyurethanes containing sulfonate groups, and polyester amides containing a group chosen from sulfonate groups and carboxyl groups. In one embodiment of the invention, hydrophilic protective colloids containing groups chosen from ionic and ion-forming groups are employed.

In one embodiment of the invention, the at least one polymer in an aqueous dispersion may be formed from at least one monomer chosen from styrene, methyl methacrylate, n-butyl (meth)acrylate, tert-butyl (meth)acrylate, acrylic acid, methacrylic acid, crotonic acid and itaconic acid.

Non-limiting examples of the at least one polymer in an aqueous dispersion may comprise, for example, those formed with styrene; those formed with styrene and at least one monomer chosen from methyl methacrylate, n-butyl (meth)acrylate, and tert-butyl (meth)acrylate; those formed with styrene, at least one monomer chosen from methyl methacrylate, n-butyl (meth)acrylate, and tert-butyl (meth)acrylate, and at least one monomer chosen from methacrylic acid, crotonic acid and itaconic acid; and those formed with styrene and at least one monomer chosen from methacrylic acid, crotonic acid and itaconic acid.

In one embodiment of the invention, the at least one polymer may be formed from at least 0.2% by weight relative to the total weight of monomers forming the at least one polymer, of at least one monomer of formula (I):

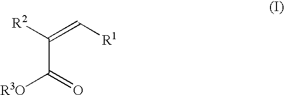

wherein R¹ and R² which may be identical or different, are each chosen from hydrogen and methyl groups, and R³ is chosen from cyclic, linear, and branched (C₉-C₃₀) alkyl groups, such as for example, (C₁₂-C₂₂) alkyl groups. In another embodiment, the at least one monomer of formula (I) comprises, for example, from 0.2% to 50% by weight relative to the total weight of monomers forming the at least one polymer, such as, from 2% to 50%, further from 0.6% to 25%, and further still from 3% to 25% by weight relative to the total weight of monomers forming the at least one polymer.

In one embodiment of the invention, R³ is chosen from linear (C₉-C₃₀) alkyl groups.

In another embodiment of the invention R³ is chosen from cyclic, linear, and branched (C₁₂-C₂₂)alkyl groups.

Non-limiting representatives of monomers of formula (I) may be chosen, for example, from esters of (meth)acrylic acid and (C₁₂-C₂₂) alcohols, such as lauryl (meth)acrylate and stearyl (meth)acrylate, esters of methylmethacrylic acid and (C₁₂-C₂₂) alcohols, and esters of crotonic acid and (C₁₂-C₂₂) alcohols.

In one embodiment of the invention, the proportion of styrene and said at least one monomer of formula (I) forming said at least one polymer ranges, for example, from 15 to 80% by weight relative to the total weight of monomer forming said at least one polymer, such as, from 30 to 60% by weight relative to the total weight of monomer forming said at least one polymer.

The at least one polymer in aqueous dispersion (called the principal polymer) may be an emulsion polymer (i.e., “the principal emulsion polymer”) comprising particles containing several domains of polymer, such as for example, particles containing several layers of polymer (also termed multilayer particles). These multilayer particles may comprise one or several polymers. In one embodiment of the invention, the multilayer particles comprise at least two polymers, a first polymer and a second polymer. The first polymer and the second polymer may each form a domain of the particles, such as for example, a layer of polymer.

In one embodiment of the invention, the first polymer present in the particles of the principal polymer can be formed from:

at least one monomer (i) having at least one group chosen from ionic groups and ion-forming groups, wherein said monomer (i) may be present in an amount ranging from 5 to 50 parts by weight, such as for example, from 8 to 30 parts by weight relative to the total weight of monomer forming said first polymer, and

at least one neutral monomer (ii), wherein said neutral monomer (ii) may be present in an amount ranging from 50 to 95 parts by weight, such as, for example, from 70 to 92 parts by weight relative to the total weight of monomer forming said first polymer.

The second polymer present in the particles of the principal polymer may be formed from at least one neutral monomer.

The at least one principal emulsion polymer may be prepared by emulsion polymerization of a mixture of monomers forming the second polymer, in the presence of the first polymer. This first polymer, as a general rule, may act as a protective colloid during the emulsion polymerization. In this way particles of polymer comprising two polymer domains, such as two polymer layers (for example, a core made up of the second polymer and a shell made up of the first polymer type) may be obtained.

The terms “first” and “second” polymer are used in the description for simplicity and do not imply in any way that more than two polymers may not be used in the multilayer particles described above.

Non-limiting representatives of monomer (i) having at least one group chosen from ionic groups and ion-forming groups may be chosen, for example, from anionic monomers, cationic monomers and amphoteric monomers.

According to one embodiment of the invention, the ion-forming groups may be converted into ionic groups by processes such as protonation/deprotonation and quaternization.

The ionic monomers can be wholly or partly neutralized.

According to one mode of implementation of the invention, the first polymer may comprise anionic and cationic monomers, it being possible for these two types of monomers to be present in equimolar quantity one relative to the other. In another embodiment, one of these two types of monomers is present in molar excess relative to the other type of monomer such that, overall, the first polymer is an ionic polymer chosen from anionic and cationic polymers. The first polymer may impart at least one of the following desirable properties to the at least one polymer: it may further the adhesion of the at least one polymer to the keratinic materials and it may further the stability of the aqueous dispersion of the at least one polymer.

Non-limiting examples of anionic or acidic monomers may be chosen, for example, from ethylenically unsaturated monocarboxylic acids and dicarboxylic acids such as those having 3 to 6 carbon atoms, polymerizable carboxylic acid derivatives and copolymerizable carboxylic acid derivatives, such as for example, those chosen from (meth)acrylic acid, crotonic acid, maleic acid, anhydrides and mono-esters thereof, fumaric acid, mono-ester of itaconic acid, itaconic acid, ethylenically unsaturated monomers containing at least one sulfonic acid group such as, for example, styrenesulfonic acid, vinyl-sulfonic acid, 2-acrylamido-2-methylpropanesulfonic acid and salts thereof, ethylenically unsaturated monomers comprising at least one group chosen, for example, from a phosphonic acid group and a phosphoric acid group such as vinyl-phosphonic acid, and mono-esters of phosphoric acid, and polymerizable alcohols, such as for example, butanediol monoacrylate and hydroxyethyl methacrylate.

Non-limiting representatives of anionic monomers may be chosen, for example, from ethylenically unsaturated monocarboxylic acids and ethylenically unsaturated dicarboxylic acids such as acrylic acid, methacrylic acid and crotonic acid.

Non-limiting representatives of cationic, or basic, monomers may be chosen, for example, from esters of (meth)acrylic acid, amides of amino alcohols, diamines, such as for example, dialkylaminoalkyl (meth)acrylates and dialkylaminoalkyl (meth)acrylamides, chosen, for example, from N,N-dimethylaminoethyl (meth)acrylate, N,N-dimethylaminoethyl methacrylate, and N,N-dimethylaminopropyl acrylamide, dialkylaminostyrenes, such as for example, N,N-di-methylaminostyrene and N,N-dimethylaminomethylstyrene, and vinylpyridine, such as for example, 4-vinyl-pyridine, 2-vinylpyridine, and 1-vinylimidazole. These cationic monomers can be quaternized with known quaternization reagents, such as for example, those chosen from alkyl halides, benzyl halides and dialkyl sulphates.

Non-limiting representatives of amphoteric monomers may be chosen, for example, from N-(3-sulphopropyl)-N-methacryloyloxyethyl-N,N-dimethyl-ammonium-betaine and N-carboxymethyl-N-methacryloyloxyethyl-N,N-dimethyl-ammonium-betaine.

It should be pointed out that the ionic groups, the acid groups, and tertiary amine groups may also be modified by salt formation or quaternization reactions.

The neutral monomers are monomers without ionic or ion-forming groups. The neutral monomers may also be chosen from monomers of formula (I) defined above. The emulsion polymer generally may be formed with other neutral monomers, different from those of formula (I), which may be chosen from principal neutral monomers and principal neutral comonomers.

Non-limiting representatives of principal neutral monomers may be chosen, for example, from:

(C₁-C₈)alkyl (meth)acrylates, such as those chosen from methyl (meth)acrylate, ethyl (meth)acrylate, propyl (meth)acrylate, n-butyl (meth)acrylate and 2-ethylhexyl (meth)acrylate; vinyl esters of (C₁-C₁₈), such as (C₁-C₈)carboxylic acids chosen, for example, from vinyl acetate, vinyl propionate, vinyl laurate and vinyl neodecanoate; and

aromatic vinylic monomers chosen, for example, from styrene, α-methylstyrene, α-butylstyrene, alkyl (C₁-C₁₀) styrenes such as 4-butylstyrene, 4-decylstyrene, hydroxystyrene, such as, 4-hydroxystyrene.

Further non-limiting examples of principal neutral monomers are chosen from aliphatic olefins having from 2 to 8 carbon atoms and one to two ethylenically unsaturated double bonds, such as for example, butadiene, isoprene, chloroprene, ethylene, propylene and isobutylene. In one embodiment of the invention, the principal neutral monomers are chosen from aliphatic olefins containing a single ethylenic double bond.

Further non-limiting examples of the principal neutral monomers are chosen from styrene, methyl methacrylate, n-butyl (meth)acrylate, tert-butyl (meth)acrylate and isobutyl (meth)acrylate.

Non-limiting examples of principal neutral comonomers may be chosen, for example, from monomers containing hydroxyl groups, such as, (C₁-C₆) hydroxyalkyl (meth)acrylates chosen, for example, from hydroxypropyl (meth)acrylate and hydroxyethyl (meth)acrylate, optionally substituted amides of ethylenically unsaturated monocarboxylic acids and optionally substituted amides of ethylenically unsaturated dicarboxylic acids, such as for example, those chosen from acrylamide, methacrylamide, N-methylolacrylamide, N-methylolmethacrylamide, (C₁-C₁₀) N-alkyl (meth)acrylamides and (C₁-C₁₀) N,N-dialkyl (meth)acrylamides. Additionally, crosslinking monomers, such as for example, those containing two vinyl groups, can be used at the same time. In one embodiment of the invention, the crosslinking monomers are present in the second polymer.

Further non-limiting examples of principal neutral comonomers may be chosen from nitrile monomers and vinyl halide monomers. Non-limiting examples of nitrile monomers may be chosen from acrylonitrile and methacrylonitrile. The vinyl halide monomers may be chosen from ethylenically unsaturated compounds substituted with a halogen atom such as for example, chlorine, fluorine and bromine. Non-limiting examples of vinyl halide monomers may be chosen, for example, from vinyl chloride, vinylidene chloride and vinylidene fluoride.

In one embodiment of the invention, the second polymer mainly comprises neutral monomers. The second polymer may be formed with an amount of principal neutral monomers ranging from 60 to 100% by weight relative to the total weight of monomer forming the second polymer cited above and from 0 to 40% by weight of at least one monomer different from the principal neutral monomers previously defined, such as for example, at least one monomer chosen from principal neutral comonomers defined above. The second polymer may further comprise at least one ionic monomer, such as those defined above in an amount less than 5% by weight relative to the weight of the second polymer.

In one embodiment, the at least one polymer in an aqueous dispersion is chosen from polymers formed from:

—a first polymer formed from:

(a) at least one monomer (i) comprising at least one group chosen from ionic groups and ion-forming groups, wherein the at least one monomer (i) is present in an amount ranging from 5 to 40 parts by weight relative to the total weight of monomer forming the first polymer,

(b) at least one monomer chosen from monomers of formula (I):

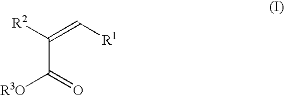

wherein R¹ and R², which may be identical or different, are each chosen from hydrogen atoms and methyl groups, and R³ is chosen from cyclic, linear, and branched (C₉-C₃₀) alkyl groups, such as for example, (C₁₂-C₂₂) alkyl groups, wherein the at least one monomer chosen from monomers of formula (I) is present in an amount ranging from, for example, 2 to 50 parts by weight, such as for example, from 10 to 30 parts by weight relative to the total weight of monomer forming the first polymer,

(c) at least one neutral monomer (ii) chosen from (C₁-C₈)alkyl (meth)acrylates, vinyl esters of (C₁-C₁₈) carboxylic acids, and aromatic vinylic monomers, wherein the at least one neutral monomer (ii) is present in an amount ranging, for example, from 10 to 93 parts by weight, such as from 40 to 85 parts by weight relative to the total weight of monomer forming the first polymer, and

(d) at least one additional monomer (d) differing from the monomers set forth in (a), (b), and (c), and wherein said at least one monomer (d) is present in an amount ranging from 0 to 40 parts by weight relative to the total weight of monomer forming the first polymer, and

a second polymer formed from:

(e) at least one neutral monomer (e) chosen, for example, from (C₁-C₈) alkyl (meth)acrylates, vinyl esters of (C₁-C₁₈) carboxylic acids, aromatic vinylic monomers, wherein the at least one monomer (e) is present in an amount ranging from 60 to 100 parts by weight relative to the total weight of monomer forming the second polymer, and

(f) at least one monomer (f) differing from the at least one neutral monomer (e), wherein the at least one monomer (f) is present in an amount ranging from, for example, from 0 to 40 parts by weight relative to the total weight of monomer forming the second polymer.

In one embodiment of the invention, the inventive film-forming composition, comprising particles of at least one polymer in an aqueous dispersion, comprises a first polymer and a second polymer, wherein the weight ratio between the first polymer and the second polymer may range, for example, from 10:90 to 60:40, such as from 30:70 to 50:50.

In one embodiment of the invention, the mean weight-average molecular weight (Mw) of the first polymer may be greater than 10,000, ranging, for example, from 20,000 to 200,000 (determined by chromatography with polystyrene as standard and tetrahydrofuran as eluent).

The first polymer can be obtained by any polymerization process, one such non-limiting example is solution polymerization.

The at least one solvent which may be utilized in the solution polymerization of the first polymer may be chosen, for example, from solvents having a boiling point below 100° C. at 100,000 Pa (1 bar), solvents which form an azeotrope with water and can be readily isolated, by distillation, from the aqueous polymer dispersion or from the polymer solution. In some embodiments of the invention, at least one auxiliary film-forming agent may be added to the solvent.

Non-limiting examples of solvents may be chosen, for example, from alcohols having up to 8 carbon atoms and ketones having up to 8 carbon atoms, such as for example, butanol, isobutanol, propanol, ethanol, methanol and methyl ethyl ketone.

The polymerization of the ethylenically unsaturated monomers can be effected by anionic polymerization and radical polymerization, such as for example, in the presence of at least one radical initiator such as peroxides. The at least one radical initiator may be present in an amount ranging, for example, from 0.2 to 5% by weight relative to the total weight of the monomers, such as, from 0.5 to 3% by weight relative to the total weight of the monomers. The polymerization temperature can be selected in a temperature ranging, for example, from 50 to 150° C., such as, from 70 to 130° C. If necessary, at least one regulator, such as for example, those chosen from mercaptoethanol, tertiary dodecylmercaptan, ethylhexyl thioglycolate and diisopropyl xanthogen sulfide, can be added. The at least one regulator may be present in an amount ranging, for example, from 0% to 3% by weight relative to the total weight of the monomers.

The preparation of the first polymer can be effected in one or several stages. In one process, for example, a polymer having a high acid content is formed in a first stage and a polymer having a lower acid content is formed in the second stage, as is described, for example, in EP-A-320865, the disclosure of which relating to the polymer preparation is specifically incorporated by reference, herein. The monomers can be introduced at various places in the process, such as for example, at the time of the polymerization or through continuous addition.

The first polymer may be obtained dispersed or in solution in the at least one solvent. The dry matter content of the first polymer may range, for example, from 50 to 95% by weight, such as, from 60 to 85% by weight relative to the total weight of the dispersion or solution in the at least one solvent.

For the production of the emulsion polymer, the emulsion polymerization may be effected in the presence of the first polymer. The emulsion polymerization is generally effected, in the presence of at least one water-soluble initiator, at a temperature ranging, for example, from 30° C. to 95° C. Non-limiting representatives of at least one water-soluble initiator, may be chosen, for example, from sodium persulfate, potassium persulfate, ammonium persulfate, tert-butyl hydroperoxide, water-soluble azo-type combinations and redox initiator systems. If H₂O₂ is used as the initiator, small quantities of heavy metal salts chosen, for example, from the salts of Cu(II) and the salts of Fe(III) may be used.

The first polymer can be introduced in water or in another aqueous medium. Additionally, the first polymer may be incorporated in water with the monomers of the second polymer undergoing polymerization during the emulsion polymerization. In one embodiment of the invention, water is added to the polymer solution and then the organic solvent used is removed by distillation.

When the first polymer contains a group chosen, for example, from acid groups and anhydride groups, these groups may be neutralized wholly or partly by means of at least one neutralizing agent. The neutralization may be conducted either before or during introduction of the first polymer into the aqueous phase.

Non-limiting examples of suitable at least one neutralizing agent may be chosen, for example, from inorganic bases, such as sodium carbonate and potassium carbonate, ammonia, organic bases, such as for example, amino alcohols such as, 2-amino-2-methyl-1-propanol (AMP), triethanolamine, triisopropylamine (TIPA), mono-ethanolamine, diethanolamine, tri[(2-hydroxy)-1-propyl]amine, 2-amino-2-methyl-1,3-propanediol (AMPD) and 2-amino-2-hydroxymethyl-1,3-propanediol and diamines, such as for example, lysine.

For the emulsion polymerization, while it may generally not be necessary to use other agents in addition to the first polymers, at least one additional agent chosen from emulsifiers, protective colloids and dispersion aids, may optionally be added.

Before and after the emulsion polymerization, at least one additive may be added to lower the viscosity. Non-limiting examples of at least one additive, may be chosen, for example, from salts of organic acids and salt of organic bases, such as, lysine hydrochloride and sodium citrate.

In the film-forming cosmetic composition according to the invention, the at least one polymer in an aqueous dispersion can be present in an amount ranging, for example, from 0.1 to 60% by weight relative to the total weight of the composition, such as, from 1% to 50% by weight and further still, from 5% to 40% by weight relative to the total weight of the composition.

The first organic solvent which may be present in the composition according to the invention, also called the coalescing agent, furthers the coalescence of the particles of polymer in an aqueous dispersion. In one embodiment of the invention, the first organic solvent has a molecular weight less than or equal to 200, such as, less than or equal to 160; further, the molecular weight ranges from 50 to 200, and further still, the molecular weight ranges from 50 to 160. The first organic solvent may have a boiling point ranging, for example, from 120° C. to 250° C., such as, from 130° C. to 230° C.

Non-limiting examples of the first organic solvent may be chosen, for example, from:

ethers of propylene glycol chosen, for example, from propylene glycol n-butyl ether, propylene glycol t-butyl ether, propylene glycol n-propyl ether and propylene glycol phenyl ether,

ethers of dipropylene glycol chosen, for example, from dipropylene glycol n-butyl ether, dipropylene glycol methyl ether, dipropylene glycol t-butyl ether and dipropylene glycol n-propyl ether, and

compounds chosen, for example, from propylene glycol methyl ether acetate, propylene glycol diacetate, methyl lactate, ethyl lactate, isopropyl lactate and butyl lactate.

The first organic solvent may be present in the film-forming cosmetic composition according to the invention in an amount sufficient to obtain a film deposited on the keratinic material, such as on nails. In one embodiment of the invention, the first organic solvent in the film-forming cosmetic composition is present in an amount ranging, for example, from 0.05% to 10% by weight relative to the total weight of the composition, such as from 0.1% to 8% by weight relative to the total weight of the composition.

The second organic solvent present in the composition, also called the plasticizer, makes it possible to plasticize the at least one polymer in an aqueous dispersion. In one embodiment of the invention, the second organic solvent has a molecular weight greater than 200, such as greater than or equal to 230, and further still, greater than or equal to 250. In one particular embodiment, the molecular weight of the second organic solvent can be less than or equal to 600, such as less than or equal to 500.

The second organic solvent may have a boiling point, for example, greater than or equal to 140° C., such as, greater than or equal to 160° C.; and further still, the boiling point is less than 500° C.

Non-limiting examples of the second organic solvent may be chosen, for example, from:

adipates chosen, for example, from diethyl adipate, dibutyl adipate, diisobutyl adipate and diisopropyl adipate,

sebacates chosen, for example, from dimethyl sebacate, diethyl sebacate and dibutyl sebacate,

citrates chosen, for example, from triethyl citrate, acetyltriethyl citrate and acetyltributyl citrate, and

phthalates chosen, for example, from diethyl phthalate, dibutyl phthalate and dioctyl phthalate.

The second organic solvent can be present in the composition according to the invention in a quantity sufficient to plasticize the polymer film deposited on the keratinic material. In one embodiment of the invention, the second organic solvent may range, for example, from 0.05% to 20% by weight relative to the total weight of the composition, such as from 0.1% to 10% by weight relative to the total weight of the composition.

In one embodiment of the invention, the film-forming cosmetic composition according to the invention does not contain 7.50% by weight of ethoxydiglycol and does contain less than 0.15% by weight of propylene glycol.

The film-forming cosmetic composition according to the invention may further comprise at least one coloring material chosen, for example, from water-soluble colorants, powder coloring materials, such as, pigments, nacreous pigments and flakes well known to the skilled person. The at least one coloring material may be present in the film-forming cosmetic composition in accordance with the invention, in an amount ranging, for example, from 0.01% to 50% by weight relative to the total weight of the composition, such as from 0.01% to 30% by weight relative to the total weight of the composition.

The film-forming cosmetic composition according to the invention may further comprise at least one thickening agent chosen, for example, from clays swelling in water such as hectorites and bentonites, aggregating thickeners such as associative polyurethanes, and associative acrylate polymers, and water-soluble cellulosic thickeners such as hydroxyethylcellulose. The at least one thickening agent may be present in an amount ranging, for example, from 0.1% to 5% by weight relative to the total weight of the composition.

The film-forming cosmetic composition according to the invention may further comprise at least one cosmetic additive known to the skilled person capable of being incorporated a cosmetic composition of this type. Non-limiting examples of the at least one cosmetic composition may be chosen, for example, from fillers, spreading agents, wetting agents, dispersing agents, anti-foam agents, preservatives, UV filters, active agents, surfactants, hydrating agents, perfumes, neutralizing agents, stabilizers and antioxidants.

Needless to say, a person skilled in the art will take care to select at least one of any of these optional additional compounds and the amount of any of these optional additional compounds, such that the advantageous properties of the composition according to the invention are not substantially adversely affected by the addition envisaged.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Any numerical value inherently contains certain errors necessarily resulting from the standard deviation found in their respective testing measurements.

The following example is intended to illustrate the invention without in anyway limiting the scope thereof.

### EXAMPLE 1

### A. Preparation of the Polymerizate Constituting the First Polymer Domain by Solution Polymerization

136 g of isopropanol were heated at 85° C. with stirred under a nitrogen atmosphere in a glass flask equipped with a reflux condenser, an armatured stirring device, a tap-funnel and a thermostatted oil-bath. Then a mixture comprising 240 g of n-propanol and 21.3 g of tert-butyl perpivalate (75%) was added over 5 hours. 15 minutes after the start of the preceding stage, a mixture of monomers comprising 14.4 g of acrylic acid, 21.6 g of n-butyl acrylate, 28.8 g of lauryl acrylate and 79.2 g of methyl methacrylate was added over a 3 hours and 30 minute time period. The polymer solution was then cooled to 80° C. and neutralized in 30 minutes with 75.6 g of a 25% by weight aqueous solution of ammonia. The mixture was again stirred for 30 minutes. The polymer solution was dispersed by addition of 1200 g of water over one hour and then the mixture was heated to a temperature of up to 100° C. 800 g of distillate was recovered. An aqueous polymer solution containing 24.9% by weight of dry matter was thus obtained. This polymer has a K value (3 g of dry matter in 100 ml of acetone) of 34.2. The K value (or Fikentscher constant) was calculated from the viscosity of the polymer in solution and as explained in the specialist literature, for example H G Elias, macromolecule, vol.1, Hüthig & Wepf, Heidelberg 1990, page 98f, the disclosure of which relating to the determination of the K value is specifically incorporated by reference, herein.

### B. Preparation of the Polymerizate in Several Stages by Emulsion Polymerization of a Mixture of Monomers in the Presence of the Polymerizate Constituting the First Polymer Domain

1285 g of polymer solution obtained in the previous stage a) was heated to 85° C. with stirring under a nitrogen atmosphere. Next a mixture comprising 38 g of water, 0.016 g of CuSO₄.5H₂O and 1.6 g of L-lysine hydrochloride was incorporated over 10 minutes. 26.7 g of a 12% aqueous solution of hydrogen peroxide by weight in water was incorporated at 85° C. Then, over 2 hours, a mixture of monomers comprising 280 g of styrene, 60 g of n-butyl acrylate and 140 g of tert-butyl acrylate and also 6.4 g of L-lysine hydrochloride and 53.6 g of water was added, and, over 2 and a half hours, 106.7 g of a 12% aqueous solution of hydrogen peroxide by weight in water. The mixture was then stirred for 1 hour at 85° C. and allowed to cool to ambient temperature while a mixture comprising 120 g of water and 80 g of silicone surfactant (CAS No. 71965-38-3) was added. After cooling, an aqueous dispersion of polymer particles containing 40.45% of dry matter was obtained. The polymer had a minimum film-forming temperature of 27° C. and a glass transition temperature of 55° C. The polymer particles had a maximum size of 85 nm and an arithmetic mean size of 94 nm.

To measure the T_{g}, the polymer sample was heated from the ambient temperature to 120° C., then the polymer was cooled to −60° C. and then again heated to 120° C. The T_{g} was measured in the course of the stage of heating from −60° C. to 120° C. All the heating and cooling stages took place at a heating rate of 20° C./min.

The particle size distribution was determined with a spectroscope, model Autosizer 2c from the Malvern company. Using this device, a determination of the total volume of all the particles of one class relative to the diameter of the particles was obtained.

### C. Preparation of a Nail Varnish

A nail varnish having the following composition was prepared:

| | | |
|---|---|---|
| aqueous polymer dispersion produced in step B, above | 35 | g |
| diethyl sebacate | 1.5 | g |
| propylene glycol methyl ether acetate | 0.5 | g |
| propylene glycol n-butyl ether | 3 | g |
| magnesium aluminium silicate (laponite XLS from Laporte) | 0.9 | g |
| pigments | 1.5 | g |
| water qsp | 100 | g |

A nail varnish which is easily applied to the nails and after drying forms a glossy, non-sticky film was obtained. The varnish was easily cleaned off with a standard commercial cleanser.

What is claimed is:

1. A film-forming cosmetic composition comprising:
particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and
at least two organic solvents wherein:
a first organic solvent has a molecular weight less than or equal to 200 and a boiling point, measured at ambient pressure, ranging from 100° C. and 300° C., and
a second organic solvent has a molecular weight greater than 200 and a boiling point, measured at ambient pressure, greater than or equal to 120° C.

2. A film-forming cosmetic composition according to claim 1, wherein said at least one polymer has a T_{g}−MFT≧12° C.

3. A film-forming cosmetic composition according to claim 1, wherein said at least one polymer has a T_{g}−MFT≧16° C.

4. A film-forming cosmetic composition according to claim 1, wherein said at least one polymer has a T_{g}−MFT≧18° C.

5. A film-forming cosmetic composition according to claim 1, wherein said at least one polymer has a T_{g}−MFT ranging from 8° C. to 25° C.

6. A film-forming cosmetic composition according to claim 1, wherein said at least one polymer has a T_{g} ranging from 40° C. to 65° C.

7. A film-forming cosmetic composition according to claim 1, wherein said particles have a size ranging from 50 to 200 nm.

8. A film-forming cosmetic composition according to claim 7, wherein said particles have a size ranging from 80 to 150 nm.

9. A film-forming cosmetic composition comprising: a film-forming composition comprising:
at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and
at least two organic solvents wherein:
a first organic solvent has a molecular weight less than or equal to 200 and a boiling point, measured at ambient pressure, ranging from 100° C. and 300° C., and
a second organic solvent has a molecular weight greater than 200 and a boiling point, measured at ambient pressure, greater than or equal to 120° C.

10. A film-forming cosmetic composition according to claim 1, wherein said at least one polymer is formed by polymerization of at least one ethylenically unsaturated monomer chosen from (C₁-C₃₀) alkyl (meth)acrylates, vinyl esters, and aromatic vinylic monomers.

11. A film-forming cosmetic composition according to claim 10, wherein said aromatic vinylic monomer is styrene.

12. A film-forming cosmetic composition according to claim 10, wherein said at least one polymer is formed by polymerization of styrene with at least one monomer chosen from methyl methacrylate, n-butyl (meth)acrylate and tert-butyl (meth)acrylate.

13. A film-forming composition according to claim 11, wherein said at least one polymer is formed by polymerization of styrene with at least one monomer chosen from acrylic acid, methacrylic acid, crotonic acid and itaconic acid.

14. A film-forming cosmetic composition according to claim 1, wherein said at least one polymer is formed from styrene, at least one monomer chosen from methyl methacrylate, n-butyl (meth)acrylate, and tert-butyl (meth)acrylate, and at least one monomer chosen from acrylic acid, methacrylic acid, crotonic acid and itaconic acid.

15. A film-forming cosmetic composition according to claim 1, wherein said at least one polymer is formed from at least 0.2% by weight, relative to the total weight of monomers forming said at least one polymer, of at least one monomer chosen from monomers of formula (I): 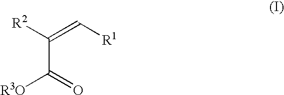
wherein R¹ and R² which may be identical or different, are each chosen from hydrogen and methyl groups, and R³ is chosen from cyclic, linear, and branched (C₉-C₃₀) alkyl groups.

16. A film-forming cosmetic composition according to claim 15, wherein said monomers of formula (I) are present in said at least one polymer in an amount ranging from 0.2 to 50% by weight relative to the total weight of monomers forming said at least one polymer.

17. A film-forming cosmetic composition according to claim 16, wherein said monomers of formula (I) are present in said at least one polymer in an amount ranging from 0.6 to 25% by weight relative to the total weight of monomers forming said at least one polymer.

18. A film-forming cosmetic composition according to claim 17, wherein said monomers of formula (I) are present in said at least one polymer in an amount ranging from 3 to 25% by weight relative to the total weight of monomers forming said at least one polymer.

19. A film-forming cosmetic composition according to claim 15, wherein R³ is chosen from cyclic, linear, and branched (C₁₂-C₂₂)alkyl groups.

20. A film-forming cosmetic composition according to claim 15, wherein R³ is chosen from linear (C₉-C₃₀)alkyl groups.

21. A film-forming cosmetic composition according to claim 15, wherein said monomers of formula (I) are chosen from esters of (meth)acrylic acid and (C₁₂-C₂₂) alcohols, esters of methylmethacrylic acid and (C₁₂-C₂₂) alcohols, and esters of crotonic acid and (C₁₂-C₂₂) alcohols.

22. A film-forming cosmetic composition according to claim 21, wherein said esters of (meth)acrylic acid and (C₁₂-C₂₂) alcohols are chosen from lauryl (meth)acrylate and stearyl (meth)acrylate.

23. A film-forming cosmetic composition according to claim 1, wherein said at least one polymer is formed from styrene and at least one monomer of formula (I): 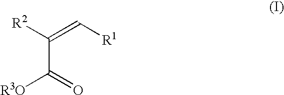
wherein R¹ and R² which may be identical or different, are each chosen from hydrogen and methyl groups, and R³ is chosen from cyclic, linear, and branched (C₉-C₃₀) alkyl groups.

24. A film-forming cosmetic composition according to claim 23, wherein the proportion of styrene and said at least one monomer of formula (I) forming said at least one polymer ranges from 15 to 80% by weight relative to the total weight of monomer forming said at least one polymer.

25. A film-forming cosmetic composition according to claim 24, wherein the proportion of styrene and said at least one monomer of formula (I) forming said at least one polymer ranges from 30 to 60% by weight relative to the total weight of monomer forming said at least one polymer.

26. A film-forming cosmetic composition according to claim 1, wherein said particles of at least one polymer are particles comprising at least two polymer domains.

27. A film-forming cosmetic composition according to claim 26, wherein said at least one polymer comprises:
a) a first polymer formed from at least one monomer chosen from:
i) at least one monomer (i) comprising at least one group chosen from ionic groups and ion-forming groups, wherein said at least one monomer (i) is present in an amount ranging from 5 to 50 parts by weight relative to the total weight of monomer forming said first polymer, and
ii) at least one neutral monomer (ii), wherein said neutral monomer (ii) is present in an amount ranging from 50 to 95 parts by weight relative to the total weight of monomer forming said first polymer, and
b) a second polymer formed from at least one neutral monomer.

28. A film-forming cosmetic composition according to claim 27, wherein said monomer (i) is chosen from anionic monomers, cationic monomers, and amphoteric monomers.

29. A film-forming cosmetic composition according to claim 28, wherein said anionic monomers are chosen from ethylenically unsaturated monocarboxylic acids, ethylenically unsaturated dicarboxylic acids, and ethylenically unsaturated monomers comprising at least one group chosen from a sulfonic acid group, a phosphonic acid group and a phosphoric acid group.

30. A film-forming cosmetic composition according to claim 29, wherein said ethylenically unsaturated monocarboxylic acids are chosen from acrylic acid, methacrylic acid and crotonic acid.

31. A film-forming cosmetic composition according to claim 28, wherein said cationic monomers are chosen from esters of (meth)acrylic acid, amides of amino alcohols, diamines chosen from dialkylaminoalkyl (meth)acrylates and dialkylaminoalkyl (meth)acrylamides, dialkylaminostyrenes, and vinylpyridines.

32. A film-forming cosmetic composition according to claim 28, wherein said amphoteric monomers are chosen from N-(3-sulphopropyl)-N-methacryloyloxyethyl-N,N-dimethyl-ammonium-betaine and N-carboxymethyl-N-methacryloyloxyethyl-N,N-dimethyl-ammonium-betaine.

33. A film-forming cosmetic composition according to claim 27, wherein said neutral monomers are chosen from:
monomers of formula (I): 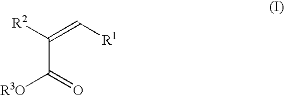
wherein R¹ and R² which may be identical or different, are each chosen from hydrogen and methyl groups, and R³ is chosen from cyclic, linear, and branched (C₉-C₃₀) alkyl groups.

34. A film-forming cosmetic composition according to claim 27, wherein said at least one polymer is further formed from neutral monomers chosen from (C₁-C₈)alkyl (meth)acrylates, vinyl esters of (C₁-C₁₈)carboxylic acids, aromatic vinylic monomers, and aliphatic olefins having from 2 to 8 carbon atoms and one to two ethylenically unsaturated double bonds.

35. A film-forming cosmetic composition according to claim 34, wherein said neutral monomers are chosen from styrene, methyl methacrylate, n-butyl (meth)acrylate, tert-butyl (meth)acrylate and isobutyl (meth)acrylate.

36. A film-forming cosmetic composition according to claim 27, wherein said at least one polymer is further formed from neutral comonomers chosen from monomers containing hydroxyl groups, optionally substituted amides of ethylenically unsaturated monocarboxylic acids, optionally substituted amides of ethylenically unsaturated dicarboxylic acids, nitrile monomers and vinyl halide monomers.

37. A film-forming cosmetic composition according to claim 27, wherein said first polymer is formed with a monomer (i) having at least one group chosen from wholly and partially neutralized ionic groups.

38. A film-forming cosmetic composition according to claim 27, wherein said at least one polymer is formed from:
a first polymer formed from:
(a) at least one monomer (i) comprising at least one group chosen from ionic groups and ion-forming groups, wherein said at least one monomer (i) is present in an amount ranging from 5 to 40 parts by weight relative to the total weight of monomer forming said first polymer,
(b) at least one monomer chosen from monomers of formula (I): 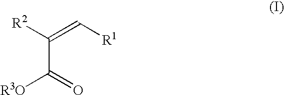
wherein R¹ and R² which may be identical or different, are each chosen from hydrogen and methyl groups, and R³ is chosen from cyclic, linear, and branched (C₉-C₃₀) alkyl groups, wherein said at least one monomer chosen from monomers of formula (I) is present in an amount ranging from 2 to 50 parts by weight relative to the total weight of monomer forming said first polymer,
(c) at least one neutral monomer (ii) chosen from (C₁-C₈)alkyl (meth)acrylates, vinyl esters of (C₁-C₁₈) carboxylic acids, and aromatic vinylic monomers, wherein said at least one neutral monomer (ii) is present in an amount ranging from 10 to 93 parts by weight relative to the total weight of monomer forming said first polymer, and
(d) at least one additional monomer (d) differing from said at least one monomer (i), said at least one neutral monomer (ii) and said at least one monomer chosen from the monomers of formula (I), wherein said at least one monomer (d) is present in an amount ranging from 0 to 40 parts by weight relative to the total weight of monomer forming said first polymer, and a second polymer formed from:
(e) at least one neutral monomer (e) chosen from (C₁-C₈) alkyl (meth)acrylates, vinyl esters of (C₁-C₁₈) carboxylic acids, aromatic vinylic monomers, wherein said at least one monomer (e) is present in an amount ranging from 60 to 100 parts by weight relative to the total weight of monomer forming said second polymer, and
(f) at least one monomer (f) differing from said at least one neutral monomer (e), wherein said at least one monomer (f) is present in an amount ranging from 0 to 40 parts by weight relative to the total weight of monomer forming said second polymer.

39. A film-forming cosmetic composition according to claim 38, wherein said at least one monomer of formula (I) is present in an amount ranging from 10 to 30 parts by weight relative to the total weight of monomer forming said first polymer.

40. A film-forming cosmetic composition according to claim 38, wherein said at least one neutral monomer (ii) is present in an amount ranging from 40 to 85 parts by weight relative to the total weight of monomer forming said first polymer.

41. A film-forming cosmetic composition according to claim 27, wherein the weight ratio of the first polymer to the second polymer ranges from 10:90 to 60:40.

42. A film-forming cosmetic composition according to claim 41, wherein the weight ratio of the first polymer to the second polymer ranges from 30:70 to 50:50.

43. A film-forming cosmetic composition according to claim 1, wherein said at least one polymer is present in an amount ranging from 0.1% to 60% by weight relative to the total weight of the composition.

44. A film-forming cosmetic composition according to claim 1, wherein said at least one polymer is present in an amount ranging from 1% to 50% by weight relative to the total weight of the composition.

45. A film-forming cosmetic composition according to claim 1, wherein said at least one polymer is present in an amount ranging from 5% to 40% by weight relative to the total weight of the composition.

46. A film-forming cosmetic composition according to claim 1, wherein said first organic solvent has a boiling point ranging from 120° C. to 250° C.

47. A film-forming cosmetic composition according to claim 1, wherein said first organic solvent has a boiling point ranging from 130° C. to 230° C.

48. A film-forming cosmetic composition according to claim 1, wherein said first organic solvent is chosen from ethers of propylene glycol, ethers of dipropylene glycol, and compounds chosen from propylene glycol methyl ether acetate, propylene glycol diacetate, methyl lactate, ethyl lactate, isopropyl lactate and butyl lactate.

49. A film-forming cosmetic composition according to claim 48, wherein said ethers of propylene glycol are chosen from propylene glycol n-butyl ether, propylene glycol t-butyl ether, propylene glycol n-propyl ether, and propylene glycol phenyl ether, and said ethers of dipropylene glycol are chosen from dipropylene glycol n-butyl ether, dipropylene glycol methyl ether, dipropylene glycol t-butyl ether, and dipropylene glycol n-propyl ether.

50. A film-forming cosmetic composition according to claim 1, wherein said first organic solvent is present in an amount ranging from 0.05% to 10% by weight relative to the total weight of the composition.

51. A film-forming cosmetic composition according to claim 50, wherein said first organic solvent is present in an amount ranging from 0.1% to 8% by weight relative to the total weight of the composition.

52. A film-forming cosmetic composition according to claim 1, wherein said second organic solvent has a boiling point greater than or equal to 140° C.

53. A film-forming cosmetic composition according to claim 1, wherein said second organic solvent has a boiling point greater than or equal to 160° C.

54. A film-forming cosmetic composition according to claim 1, wherein said second organic solvent has a boiling point ranging from 140 to 500° C.

55. A film-forming cosmetic composition according to claim 1, wherein said second organic solvent is chosen from adipates, sebacates, citrates, and phthalates.

56. A film-forming cosmetic composition according to claim 55, wherein said second organic solvent is chosen from diethyl adipate, dibutyl adipate, diisobutyl adipate, diisopropyl adipate, dimethyl sebacate, diethyl sebacate, dibutyl sebacate, triethyl citrate, acetyltriethyl citrate, acetyltributyl citrate, diethyl phthalate, dibutyl phthalate and dioctyl phthalate.

57. A film-forming cosmetic composition according to claim 1, wherein said second organic solvent is present in an amount ranging from 0.05% to 20% by weight relative to the total weight of the composition.

58. A film-forming cosmetic composition according to claim 57, wherein said second organic solvent is present in an amount ranging from 0.1% to 10% by weight relative to the total weight of the composition.

59. A film-forming cosmetic composition according to claim 1 further comprising at least one coloring material chosen from water-soluble colorants and powder coloring materials.

60. A film-forming cosmetic composition according to claim 59, wherein said powder coloring materials are chosen from pigments, nacreous pigments and flakes.

61. A film-forming cosmetic composition according to claim 59, wherein said at least one coloring material is present in an amount ranging from 0.01% to 50% by weight relative to the total weight of the composition.

62. A film-forming cosmetic composition according to claim 61, wherein said at least one coloring material is present in an amount ranging from 0.01% to 30% by weight relative to the total weight of the composition.

63. A film-forming cosmetic composition according to claim 1 further comprising at least one thickening agent.

64. A film-forming cosmetic composition according to claim 63, wherein said at one least thickening agent is chosen from clays swelling in water, aggregating thickeners, and water-soluble cellulosic thickeners.

65. A film-forming cosmetic composition according to claim 64, wherein said at least one thickening agent is present in an amount ranging from 0. 1% to 5% by weight relative to the total weight of the composition.

66. A film-forming cosmetic composition according to claim 1 further comprising at least one cosmetic additive.

67. A film-forming cosmetic composition according to claim 66, wherein said at least one cosmetic additive is chosen from fillers, spreading agents, wetting agents, dispersants, anti-foam agents, preservatives, UV filters, active agents, surfactants, hydrating agents, perfumes, neutralizing agents, stabilizers and antioxidants.

68. A film-forming cosmetic composition according to claim 1 further comprising at least one compound chosen from emulsifiers and protective colloid agents.

69. A film-forming cosmetic composition according to claim 1, wherein said film-forming cosmetic composition is a making-up or cosmetic care composition for a keratinic material.

70. A film-forming cosmetic composition comprising:
particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and
at least two organic solvents wherein:
a first organic solvent has a molecular weight less than or equal to 200 and a boiling point, measured at ambient pressure, ranging from 100° C. and 300° C., and
a second organic solvent has a molecular weight greater than 200 and a boiling point, measured at ambient pressure, greater than or equal to 120° C.,
wherein said film-forming cosmetic composition is a nail varnish.

71. A method for making-up or care of a keratinic material comprising:
applying onto said keratinic material a film-forming cosmetic composition comprising:
particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and
at least two organic solvents, wherein:
a first organic solvent has a molecular weight less than or equal to 200 and a boiling point, measured at ambient pressure, ranging from 100° C. and 300° C., and
a second organic solvent has a molecular weight greater than 200 and a boiling point, measured at ambient pressure, greater than or equal to 120° C.

72. A method for forming a film comprising applying to a keratinic material a film-forming composition comprising:
at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and
at least two organic solvents wherein:
a first organic solvent has a molecular weight less than or equal to 200 and a boiling point, measured at ambient pressure, ranging from 100° C. and 300° C., and
a second organic solvent has a molecular weight greater than 200 and a boiling point, measured at ambient pressure, greater than or equal to 120° C.,
wherein the film formed on the keratinic material has at least one of the following properties: is removable with at least one compound chosen from acetone and ethyl acetate; adheres to the nail; and is glossy.

73. A method for forming a film comprising applying to a keratinic material a film-forming composition comprising:
particles of at least one polymer in an aqueous dispersion, wherein said at least one polymer has a glass transition temperature (T_{g}) ranging from 35° C. to 80° C. and a minimum film-forming temperature (MFT) such that T_{g}−MFT≧8° C.; and
at least two organic solvents wherein:
a first organic solvent has a molecular weight less than or equal to 200 and a boiling point, measured at ambient pressure, ranging from 100° C. and 300° C., and
a second organic solvent has a molecular weight greater than 200 and a boiling point, measured at ambient pressure, greater than or equal to 120° C.,
wherein the film formed on the keratinic material has at least one of the following properties: is removable with at least one compound chosen from acetone and ethyl acetate; adheres to the nail; and is glossy.

74. A method for making-up or care of a keratinic material according to claim 71, wherein said keratinic material is a human nail.

75. A method for forming a film according to claim 72, wherein said keratinic material is a human nail.

76. A method for forming a film according to claim 73, wherein said keratinic material is a human nail.
